(19) United States
(12) Patent Application Publication
(10) Pub. No.: US 2023/0087246 A1
(43) Pub. Date: Mar. 23, 2023

(54) DELIVERY SYSTEM FOR AQUEOUS ZINC ORAL CARE COMPOSITIONS WITH FLUORIDE

(71) Applicant: 3M INNOVATIVE PROPERTIES COMPANY, St. Paul, MN (US)

(72) Inventors: Yizhong WANG, Woodbury, MN (US); Tiffany T. TON, Woodbury, MN (US); Richard P. RUSIN, Woodbury, MN (US)

(21) Appl. No.: 17/759,453

(22) PCT Filed: Jan. 28, 2021
(86) PCT No.: PCT/IB2021/050677

§ 371 Date: Jul. 26, 2022

Related U.S. Application Data
(60) Provisional application No. 62/968,112, filed on Jan. 30, 2020.

(51) Int. Cl. A61K 8/69; A61C 19/06; A61Q 11/00; A61Q 17/00

(52) U.S. Cl. CPC A61K 8/69; A61C 19/063; A61Q 11/00; A61Q 17/005; A61C 2202/00; A61K 2800/87

(57) ABSTRACT

Zinc-fluoride compositions, dental articles comprising zinc-fluoride compositions, and methods for reducing bacteria on a tooth surface and/or remineralizing a tooth surface are described.

### BACKGROUND

Dental caries is a disease in which tooth decay results from interaction with acid produced by bacteria. Silver salts are known to have antibacterial properties and fluoride salts are known to remineralize tooth surfaces. Both silver and fluoride compositions have separately been shown to aid in preventing and arresting caries activities.

Silver diamine fluoride has been used to treat surfaces affected by caries; however, reaction with saliva causes the formation of silver phosphate. Silver phosphate is light sensitive and permanently stains teeth black when exposed to light. Silver fluoride (without an amine or other ligand stabilizer) is extremely unstable in aqueous solutions and quickly decomposes to form metallic silver.

Zinc salts are also known to have antibacterial properties and do not suffer from light sensitivity, i.e., zinc complexes do not turn black upon exposure to light. Together, zinc and fluoride have the potential to create a promising caries treatment. However, zinc fluoride, as well as other zinc complexes, are not soluble in aqueous solutions. In fact, zinc fluoride will often precipitate out of solution even if water-soluble zinc precursors are employed.

What is needed is a way to treat a tooth surface with an aqueous solution of zinc and fluoride. In particular, there is a need to treat interproximal tooth surfaces in an efficient, safe, and aesthetically-pleasing manner.

### SUMMARY

In one embodiment, a dental article configured to access an interproximal gap is described. The dental article may include at least one applicator, a zinc-fluoride composition, and a package to contain one or more of the at least one applicator and the zinc-fluoride composition. The zinc-fluoride composition may include a zinc carboxylate, an amine-containing ligand, a source of fluoride anion effective to provide fluoride in an amount of at least 4 wt % with respect to the weight of the zinc-fluoride composition, and water. The zinc-fluoride composition may have a pH of at least 8 and be a homogenous solution at a temperature of about 20-25° C. The package may be impermeable to water.

In one embodiment, a dental article is described. The dental article may include at least one applicator, a zinc-fluoride composition, and a package to contain one or more of the at least one applicator and the zinc-fluoride composition. The zinc-fluoride composition may include a zinc carboxylate, an amine-containing ligand, a source of fluoride anion effective to provide fluoride in an amount of at least 4 wt % with respect to the weight of the zinc-fluoride composition, and water. The zinc-fluoride composition may have a pH of at least 8 and be a homogenous solution at a temperature of about 20-25° C. The package may be impermeable to water.

In one embodiment, a method for reducing bacteria on a tooth surface and remineralizing a tooth surface is described. The method may include providing at least one applicator and a zinc-fluoride composition, contacting an effective amount of the zinc-fluoride composition to the tooth surface with the at least one applicator, and allowing the zinc-fluoride composition to contact the tooth surface for a period. The zinc-fluoride composition may include a zinc carboxylate, an amine-containing ligand, a source of fluoride anion effective to provide fluoride in an amount of at least 4 wt % with respect to the weight of the zinc-fluoride composition, and water. The zinc-fluoride composition may have a pH of at least 8 and be a homogenous solution at a temperature of about 20-25° C. The zinc-fluoride composition may reduce bacteria at the tooth surface and remineralize a tooth surface.

In one embodiment, a method for reducing bacteria on a tooth surface is described. The method may include providing at least one applicator and a zinc-fluoride composition, contacting an effective amount of the zinc-fluoride composition to the tooth surface with the at least one applicator, and allowing the zinc-fluoride composition to contact the tooth surface for a period. The zinc-fluoride composition may include a zinc carboxylate, an amine-containing ligand, a source of fluoride anion effective to provide fluoride in an amount of at least 4 wt % with respect to the weight of the zinc-fluoride composition, and water. The zinc-fluoride composition may have a pH of at least 8 and be a homogenous solution at a temperature of about 20-25° C. The zinc-fluoride composition may reduce bacteria at the tooth surface.

In one embodiment, a method for remineralizing a tooth surface is described. The method may include providing at least one applicator and a zinc-fluoride composition, contacting an effective amount of the zinc-fluoride composition to the tooth surface with the at least one applicator, and allowing the zinc-fluoride composition to contact the tooth surface for a period. The zinc-fluoride composition may include a zinc carboxylate, an amine-containing ligand, a source of fluoride anion effective to provide fluoride in an amount of at least 4 wt % with respect to the weight of the zinc-fluoride composition, and water. The zinc-fluoride composition may have a pH of at least 8 and be a homogenous solution at a temperature of about 20-25° C. The zinc-fluoride composition may remineralize the tooth surface.

In one embodiment, a kit is provided. The kit may include a dental article including a at least one applicator and a zinc-fluoride composition. Alternatively, the kit may include at least one applicator and a zinc-fluoride composition. The zinc-fluoride composition may include a zinc carboxylate, an amine-containing ligand, a source of fluoride anion effective to provide fluoride in an amount of at least 4 wt % with respect to the weight of the zinc-fluoride composition, and water. The zinc-fluoride composition may have a pH of at least 8 and be a homogenous solution at a temperature of about 20-25° C. The kit may further include a set of instructions directing a user to perform method steps for reducing bacteria at a tooth surface described herein or methods steps for remineralizing a tooth surface described herein.

### BRIEF DESCRIPTION OF DRAWINGS

FIG. 1A is an example applicator of the present disclosure.

FIG. 1B is an example applicator of the present disclosure.

FIG. 1C is an example applicator of the present disclosure.

FIG. 2 is an example package of the present disclosure.

FIG. 3A is an example dental article the present disclosure.

FIG. 3B is an example dental article of the present disclosure.

FIG. 4A is an example dental article of the present disclosure.

FIG. 4B is an example dental article of the present disclosure.

FIG. 5A is an example dental article of the present disclosure.

FIG. 5B is an example dental article of the present disclosure.

FIG. 5C is an example dental article of the present disclosure.

FIG. 6 is a flowchart illustrating a method of the present disclosure.

FIG. 7A is an example kit of the present disclosure.

FIG. 7B is an example kit of the present disclosure.

### DETAILED DESCRIPTION

The zinc-fluoride compositions described herein are effective in arresting caries activity and repairing the damage caused by caries bacteria without staining teeth. However, the zinc-fluoride composition requires water to remain effective. In the absence of sufficient water, the zinc-fluoride composition may decompose or form solid and loss of application function. The dental articles described herein may provide a way to extend the shelf-life of the zinc-fluoride composition (at a calculated estimate of about 2 years) by preventing water evaporation. Furthermore, the dental articles described herein provide a means to reach interproximal areas and areas below the gumline that may be affected by caries without having to use the composition in excess.

As used herein, “about” means±10 percent of a given value. For example, about 10 means 9 to 11.

As used herein, “zinc carboxylate” means a zinc complex having one or more carboxylate ligands, e.g., —OC(O)—R.

As used herein, “homogenous solution” refers to a visually clear or transparent solution. No particulates or cloudiness is observed in the homogenous solutions described herein. A homogenous solution excludes dispersions, suspension, and the like.

As used herein, “impermeable” is used to define a material that does not allow for the passage of water or water vapor in any appreciable amount, i.e., The Moisture Vapor Transmission Rate (MVTR) of the material should be less than 0.0005 gram per 100 square inches in 24 hrs. The impermeable packages described herein protects the zinc-fluoride composition from decomposition due to loss of water.

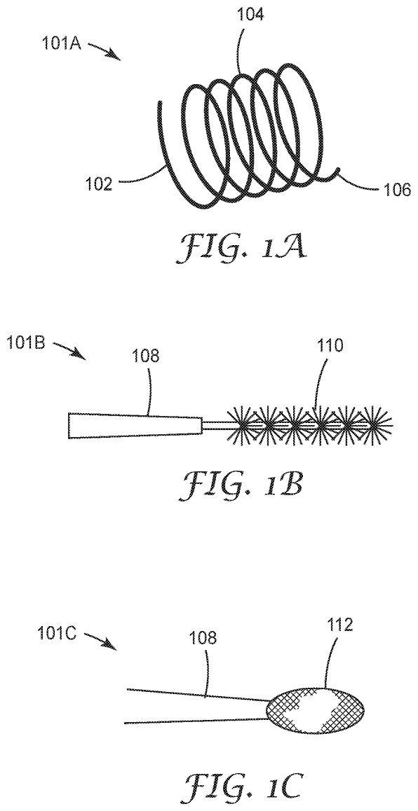

FIG. 1A illustrates an example floss applicator 101A. Floss applicator 101A may include a standard floss portion 102, a spongy portion 104, and a threading portion 106.

FIG. 1B illustrates an example microbrush applicator 101B. Microbrush applicator 101B may include a handle 108B and a brush tip 110.

FIG. 1C illustrates an example swab applicator 101C. Swab applicator 101C may include a handle 108C and a swab tip 112.

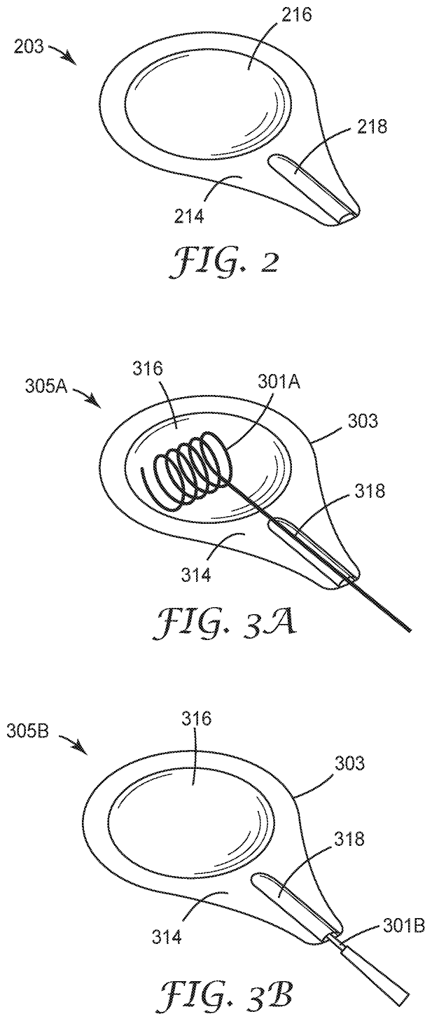

FIG. 2 illustrates an example blister-type package 203. Blister-type package 203 may include a sealed perimeter body 214 having a cavity 216 for storing a zinc-fluoride composition. Blister-type package 203 may further include an exit path 218 for expelling the zinc-fluoride composition.

FIG. 3A illustrates an example dental article 305A having a blister-type package 303. Blister-type package 303 may include a sealed perimeter body 314 having a cavity 316 for storing a zinc-fluoride composition. Blister-type package 303 may further include an exit port 318 for expelling the zinc-fluoride composition. Dental article 305A is further shown to house a floss applicator 301A within cavity 316 and that floss applicator 301A may be removed from cavity 316 via exit port 318.

FIG. 3B illustrates an example dental article 305B having a blister-type package 303. Blister-type package 303 may include a sealed perimeter body 314 having a cavity 316 for storing a zinc-fluoride composition. Blister-type package 303 may further include an exit port 318 for expelling the zinc-fluoride composition. Dental article 305B is further shown to house a microbrush applicator 301A (or swab not shown) within exit port 318.

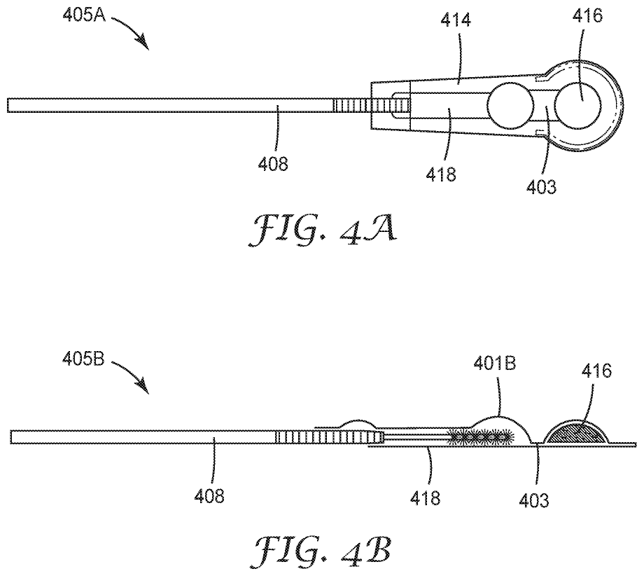

FIG. 4A illustrates an example dental article 405A. Dental article 405A may include a blister-type package 403. Blister-type package 403 may include a sealed perimeter body 414 having a cavity 416 for storing a zinc-fluoride composition. Blister-type package 403 may further include an exit path 418 for expelling the zinc-fluoride composition. Dental article 405A further shows a handle 408 of a microbrush or swab applicator.

FIG. 4A illustrates an example dental article 405A. Dental article 405A may include a blister-type package 403. Blister-type package 403 may include a sealed perimeter body 414 having a cavity 416 for storing a zinc-fluoride composition. Blister-type package 403 may further include an exit path 418 for expelling the zinc-fluoride composition. Dental article 405A further shows a handle 408 of a microbrush applicator 410B.

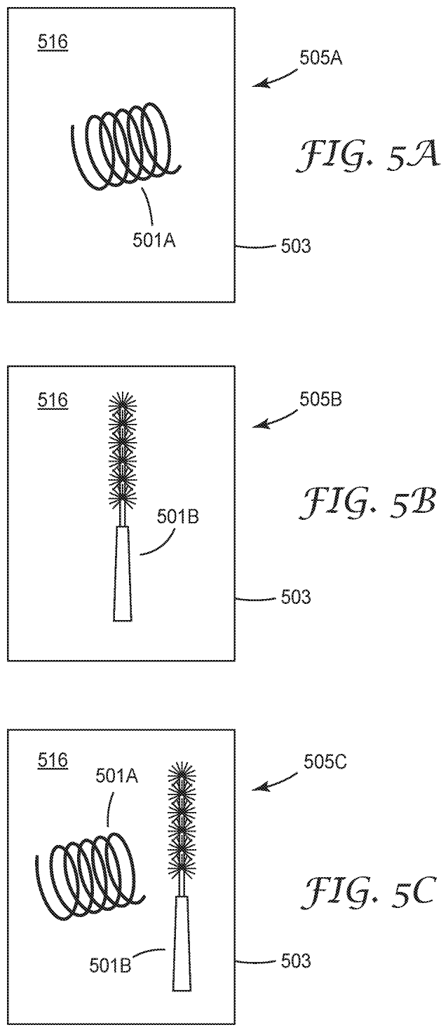

FIG. 5A illustrates an example dental article 505A. Dental article 505A may include a foil pouch-type package 503. Foil pouch-type package 503 may include a cavity 516 for housing a floss applicator 501A (or swab not shown) and the zinc-fluoride composition.

FIG. 5B illustrates an example dental article 505B. Dental article 505B may include a foil pouch-type package 503. Foil pouch-type package 503 may include a cavity 516 for housing a microbrush applicator 501B (or swab not shown) and the zinc-fluoride composition.

FIG. 5C illustrates an example dental article 505C. Dental article 505C may include a foil pouch-type package 503. Foil pouch-type package 503 may include a cavity 516 for housing a floss applicator 501A and a microbrush applicator 501B (or swab not shown) and the zinc-fluoride composition.

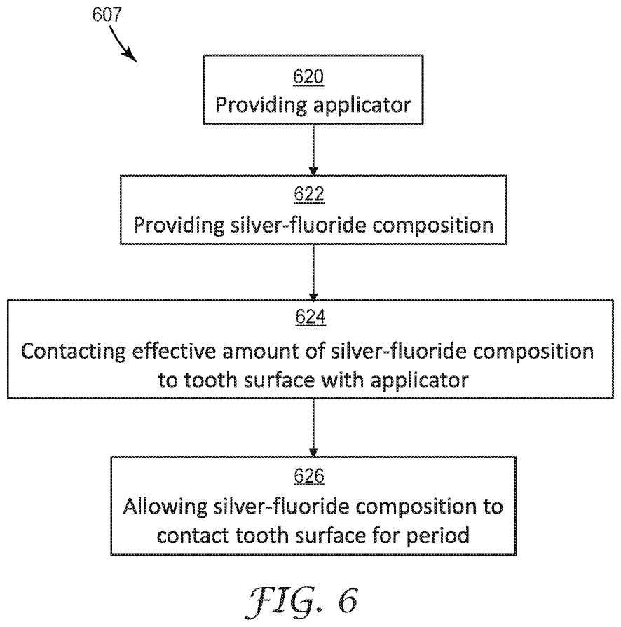

FIG. 6 illustrates a method 607 of the present disclosure. Method 607 may include 620 providing an applicator, 622 providing a zinc-fluoride composition, 624 contacting an effective amount of the zinc-fluoride composition to a tooth surface with the applicator, and 626 allowing the zinc-fluoride composition to contact the tooth surface for a period.

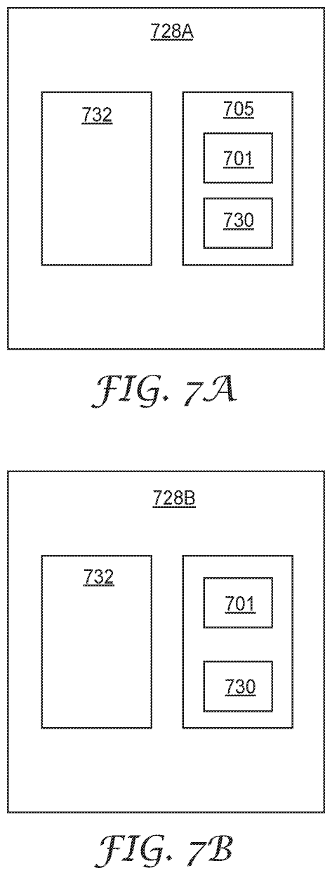

FIG. 7A illustrates an example kit 728A. Kit 728A may include a dental article 705. Dental article 705 may include an applicator 701 and a zinc-fluoride composition 730. Kit 728A may further include a set of instructions 732 directing a user to perform the steps of a method, e.g., method 607, described herein.

FIG. 7B illustrates an example kit 728B. Kit 728B may include an applicator 701 and a zinc-fluoride composition 730. Kit 728B may further include a set of instructions 732 directing a user to perform the steps of a method, e.g., method 607, described herein.

### Dental Articles

In many embodiments, a dental article or dental article configured to access an interproximal gap is described. The dental article may include at least one applicator, a zinc-fluoride composition, and a package to contain one or more of the at least one applicator and the zinc-fluoride composition. The zinc-fluoride composition may include a zinc carboxylate, an amine-containing ligand, a source of fluoride anion effective to provide fluoride in an amount of at least 4 wt % with respect to the weight of the zinc-fluoride composition, and water. The zinc-fluoride composition may have a pH of at least 8 and be a homogenous solution at a temperature of about 20-25° C. The package may be impermeable to water.

In many embodiments, the dental article includes one or more applicator described herein.

In some embodiments, at least one applicator may be loaded with the zinc-fluoride composition within the package. For example, the applicator may be stored in a solution of the zinc-fluoride composition. In another example, the zinc-fluoride composition may be absorbed on or within the applicator.

In other embodiments, the at least one applicator may be separated from the zinc-fluoride composition within the package.

In other embodiments, the at least one applicator may be loaded with the zinc-fluoride composition within the package, and the package may further include a separate compartment the zinc-fluoride composition.

In some embodiments, the dental article may include two applicators described herein.

In some embodiments, one or more applicator may be loaded with the zinc-fluoride composition within the package. In some embodiments one applicator is loaded with the zinc-fluoride composition in the package.

In some embodiments, the zinc-fluoride composition may consist essentially of the zinc carboxylate, the amine-containing ligand, the source of fluoride, and water. In fact, no thickener, silica, or polymer vehicle may be required.

In some embodiments, the zinc carboxylate may be present in an amount effective to provide zinc in a wt % with respect to the weight of the zinc-fluoride composition of about 5 wt % to about 15 wt %. For example, the zinc carboxylate may provide in zinc in a wt % with respect to the weight of the composition of about 5, 6, 7, 8, 9, 10, 11, 12, 13, 14, or 15, or a value within a range between any of the preceding values, for example, between about 8 and about 15, between about 10 and about 12, or the like.

In some embodiments, the amine-containing ligand may be present in an amount between about 5 wt % to about 30 wt % with respect to the weight of the zinc-fluoride composition. For example, the amine-containing ligand may be present in amount in wt % of about 5, 6, 7, 8, 9, 10, 12, 14, 16, 18, 20, 22, 24, 26, 28, or 30, or a value within a range between any of the preceding values, for example, between about 8 and about 16, between about 10 and about 15, or the like.

In some embodiments, the zinc carboxylate and amine-containing ligand are present in amounts to provide a mol ratio of about 1:2 to about 1:3. In some embodiments, the mol ratio is 1:2. In other embodiments, the mol ratio is greater than 1:2.

In some embodiments, the source of fluoride anion is present in an amount to provide an effective amount of fluoride in an amount of at least about 10 wt %, at least about 15 wt %, or at least about 20 wt % with respect to the weight of the zinc-fluoride composition.

In some embodiments, the source of fluoride anion is present in an amount to provide an effective amount of fluoride in an amount from about 4 wt % to about 20 wt %. For example, the source of fluoride anion may provide fluoride in an amount in wt % with respect to the weight of the zinc-fluoride composition of about 4, 6, 8, 10, 12, 14, 16, or 20, or a value within a range between any of the preceding values, for example, between about 14 and about 10, or between about 8 and about 16, or the like.

In some embodiments, the water is present in an amount of about 20 wt % to about 50 wt % with respect to the weight of the zinc-fluoride composition. For example, the water may be present in an amount of about 20, 22, 24, 26, 28, 30, 32, 34, 36, 38, 40, 42, 44, 46, 48, or 50, or a value within a range between any of the preceding values, for example between about 28 and about 50, between about 36 and about 40, or the like.

In some embodiments, the pH of the zinc-fluoride composition may be at least 8, at least 9, at least 10, at least 11, or at least 12. In some embodiments, the zinc-fluoride composition may have a pH of about 8 to about 10. In some embodiments, the pH may be 9.

In many embodiments, the zinc-fluoride compositions described herein are stable to light. In other words, the zinc-fluoride compositions are not sensitive to light and thereby do not decompose into species that cause blackening of teeth. In some embodiments, the zinc-fluoride compositions are stable to LED curing light at a wavelength of 450 nm.

### Applicator

In many embodiments, any applicator described herein may include a means for transferring a zinc-fluoride composition to a tooth surface. In some embodiments, an applicator may have an absorbent or adsorbent surface. The absorbent or adsorbent surface may include hydrophilic components. In other embodiments, an applicator may include an area configured to hold a solution via capillary action.

In some embodiments, an applicator may be selected from a floss, a microbrush, a pick, a pick including a floss (floss pick), a swab, and a combination thereof, or the like.

In some embodiments, an applicator may be selected from a floss and a floss pick.

In some embodiments, an applicator may be selected from a floss, a floss pick, and a microbrush.

In some embodiments, an applicator may be selected from a floss and a microbrush.

In some embodiments, an applicator may be a floss. The floss may be one or more of tangled, twisted, and braided.

In some embodiments, an applicator may be or include a floss. For example, a floss may include any floss known in the art, such as those made from nylon, polyester, or the like. In some embodiments, the floss may be one or more of tangled, twisted, and braided. The floss may include a coating conducive to absorbing or adsorbing an aqueous composition. In some embodiments, the floss may include a threader, e.g., a stiffened-end, a spongy floss portion, and a standard floss portion, such as used in the floss sold under the tradename Super Floss® (Oral-B; Iowa City, Iowa). The spongy floss portion may include one or more of a diameter thicker than the standard floss portion and material having a transferring capacity, e.g., absorbent, adsorbent, capillary action properties, or the like.

In some embodiments, an applicator may include a pick including a floss. The pick may include a set of prongs having a floss extending between. The floss may include any floss known in art and described herein.

In some embodiments, an applicator may be or include a microbrush. The microbrush may include fibers, bristles, or the like.

In some embodiments, more than one applicator may be used to the deliver the zinc-fluoride composition. For example, a floss may be loaded with a microbrush having a zinc-fluoride composition disposed thereon, and the floss contacted to a tooth surface. For example, a floss may be contacted to a tooth surface and a microbrush having a zinc-fluoride composition disposed thereon may load the floss in contact with the tooth surface.

### Zinc Carboxylate

In some embodiments, the zinc carboxylate is selected from one or more of zinc citrate, zinc gluconate, zinc maleate, zinc acetate, zinc fumarate, zinc adipate, and zinc propionate.

In some embodiments, the zinc carboxylate is zinc citrate.

### Amine-Containing Ligand

In some embodiments, the amine-containing ligand may include one or more of an amino acid and ammonia.

In some embodiments, the amine-containing ligand may include one or more amino acid.

In some embodiments, the amine-containing ligand is selected from one or more of histidine, isoleucine, leucine, lysine, tyrosine, tryptophan, methionine, phenylalanine, aspartic acid, glycine, arginine, glutamic acid, valine, alanine, threonine, cysteine, proline, and asparagine.

In some embodiments, the amine-containing ligand may be arginine.

In some embodiments, the amine-containing ligand is ammonia (H₃N).

In other embodiments, the amine-containing ligand may include an amine of the formula R₃N, wherein one or more R is a C₁₋₄ alkyl.

### Fluoride Anion Source

In some embodiments, the source of fluoride anion is selected from one or more of sodium fluoride, ammonium fluoride, silver fluoride, stannous fluoride, and potassium fluoride.

In some embodiments, the source of fluoride anion is selected from one or more of ammonium fluoride, silver fluoride, stannous fluoride, and potassium fluoride.

In other embodiments, the source of fluoride is not sodium fluoride.

In some embodiments, the source of fluoride anion is ammonium fluoride.

### Package

In many embodiments, the package may include any package known in the art that is impermeable to water on the order of about MVTR less than 0.0005 gram per 100 square inches in 24 hrs., MVTR less than 0.005 gram per 100 square inches in 24 hrs.

In many embodiments, the package may include a metal foil. The metal foil may include metal components selected from aluminum, copper, chromium, gold, iron, manganese, molybdenum, nickel, niobium, silver, tin, titanium, tungsten, vanadium, zinc, and a combination (alloy) thereof. In some embodiments, the metal foil may include foil packaging such as those sold by Oliver™ Healthcare Packaging, e.g, Ofoil™ 48, dispos-a-vent®, or the like.

In many embodiments, the package may include an adhesive for package sealing. The adhesive may include any heat sealant known in the art. For example, the adhesive may include polyethylene terephthalate, polyethylene, or the like.

In some embodiments, the package may be a heat-sealed foil package having a zinc-fluoride composition.

In some embodiments, the package may include a single-unit dosage of the zinc-fluoride composition.

In other embodiments, the package may include multiple-unit dosages of the zinc-fluoride composition.

In some embodiments, the package may be a heat-sealed foil package having a zinc-fluoride composition. In some embodiments, the package may further include a floss or floss pick wherein the floss or floss pick may be stored in a compartment with the zinc-fluoride composition, or in a compartment separate from the zinc-fluoride composition. In some embodiments, the package may further include a microbrush wherein the microbrush may be stored in a compartment with the zinc-fluoride composition, or in a compartment separate from the zinc-fluoride composition. In some embodiments, the package may include a floss or floss pick and a microbrush, wherein the floss or floss pick and the microbrush may be independently stored in a compartment with the zinc-fluoride composition, or in a compartment separate from the zinc-fluoride composition.

In some embodiments, the package may be a blister pack, such as those resembling L-Pop™ sold by 3M ESPE (Maplewood, Minn.). In some embodiments, the blister pack may further include a microbrush. In other embodiments, the blister pack may further include a floss within the blister pack.

In some embodiments, the package may include a one or more pieces of floss or floss picks. In other embodiments, the package may include a spool of floss for multiple-use.

In some embodiments, the package may be resealable or otherwise have an effective mechanism for preventing moisture loss after opening.

In one embodiment, the package may be a container including a spool of floss suspended in a zinc-fluoride composition. The container may include a network that separates the zinc-fluoride composition from a floss exit point such that water vapor may condense back into the container.

### Methods

In one embodiment, a method for reducing bacteria on a tooth surface and remineralizing a tooth surface is described. The method may include providing at least one applicator and a zinc-fluoride composition, contacting an effective amount of the zinc-fluoride composition to the tooth surface with the at least one applicator, and allowing the zinc-fluoride composition to contact the tooth surface for a period. The zinc-fluoride composition may include a zinc carboxylate, an amine-containing ligand, a source of fluoride anion effective to provide fluoride in an amount of at least 4 wt % with respect to the weight of the zinc-fluoride composition, and water. The zinc-fluoride composition may have a pH of at least 8 and be a homogenous solution at a temperature of about 20-25 ° C. The zinc-fluoride composition may reduce bacteria at the tooth surface and remineralize a tooth surface.

In some embodiments, the methods described herein may include any dental article described herein.

In many embodiments, the methods described herein arrest caries activity and repair damage to tooth structure caused by caries activity.

In some embodiments, the method may further include inserting an applicator onto the tooth surface, e.g., into an interproximal gap, and loading the applicator with the zinc-fluoride composition. In some embodiments, the applicator is a floss or floss pick.

In other embodiments, the method may further include inserting an applicator one the tooth surface, e.g., into an interproximal gap, wherein the applicator is pre-loaded with the zinc-fluoride composition. In some embodiments, the applicator is a floss or floss pick.

In some embodiments, the method may further include re-loading the applicator, e.g., a floss or floss pick, by one or more of submerging the applicator in the zinc-fluoride composition or applying additional zinc-fluoride composition to the applicator with a second loaded applicator, e.g., a microbrush. In some embodiments, applying additional zinc-fluoride composition to the applicator may occur while the applicator is in contact with the tooth surface.

In some embodiments, method may include contacting an effective amount of the zinc fluoride composition to the tooth surface, wherein an effective amount is at least about 30 mg the zinc-fluoride composition. In some embodiments, an effective amount is about 30 mg to about 150 mg. For example, the effective amount of zinc-fluoride composition in mg may be about 30, 40, 50, 60, 70, 80, 90, 100, 110, 120, 130 , 140 or 150, of a value in a range between any of the preceding values, for example, between about 50 and about 80, between about 30 and about 100, or the like. The effective amount may be delivered in one or more applicator loadings.

In some embodiments, the method may further include allowing the zinc-fluoride composition to contact the tooth surface for a period of about 30 to about 120 seconds. For example, the zinc-fluoride composition may be allowed to contact the tooth surface for a period in seconds of about 30, 40, 50, 60, 70, 80, 90, 100, 110, and 120, or a value between a range of any of the preceding values, e.g., between about 60 and about 90, between about 40 and about 100, or the like. When the method may include one or more applicator loading, each loading may be independently allowed to contact the tooth surface for any period described above.

In some embodiments, the applicator may be removed from the tooth surface while the zinc-fluoride composition is allowed to contact the tooth surface.

In some embodiments, the method may further include scrubbing the tooth surface during the period in which the zinc-fluoride composition contacts the tooth surface. The scrubbing may be performed with the applicator or another device.

In some embodiments, the method may further include cleaning the tooth surface prior to contacting the zinc-fluoride composition to the tooth surface. In some embodiments, the method may further include cleaning the tooth surface after allowing the zinc-fluoride composition to contact the tooth surface for a period.

In some embodiments, the method may further include drying the tooth surface prior to contacting the zinc-fluoride composition to the tooth surface. In some embodiments, the method may further include drying the tooth surface after allowing the zinc-fluoride composition to contact the tooth surface for a period.

In some embodiments, the method may further include cleaning and drying the tooth surface prior to contacting the zinc-fluoride composition to the tooth surface. In some embodiments, the method may further include cleaning and drying the tooth surface after allowing the zinc-fluoride composition to contact the tooth surface for a period.

In some embodiments, the method may further include applying a varnish after the zinc-fluoride composition is allowed to contact the tooth surface for period. The varnish may include, for example, a source of fluoride and a resin.

In some embodiments, the tooth surface contacted by the zinc-fluoride composition of the methods described herein may be any tooth surface accessible by the at least one applicator. In some embodiments, the tooth surface is an interproximal tooth surface. In other embodiments, the tooth surface is an occlusal surface. In some embodiments, the tooth surface is a buccal surface. In some embodiments, the tooth surface is a lingual surface. In some embodiments, the tooth surface is below the gumline.

In many embodiments, the methods described herein may include any applicator(s) described herein.

In many embodiments, the methods described herein may include any zinc-fluoride composition described herein, including any source of cations, anions, or the like described herein.

In many embodiments, the methods described herein may include any package(s) described herein.

### Reducing Bacteria

In one embodiment, a method for reducing bacteria on a tooth surface is described. The method may include providing at least one applicator and a zinc-fluoride composition, contacting an effective amount of the zinc-fluoride composition to the tooth surface with the at least one applicator, and allowing the zinc-fluoride composition to contact the tooth surface for a period. The zinc-fluoride composition may include a zinc carboxylate, an amine-containing ligand, a source of fluoride anion effective to provide fluoride in an amount of at least 4 wt % with respect to the weight of the zinc-fluoride composition, and water. The zinc-fluoride composition may have a pH of at least 8 and be a homogenous solution at a temperature of about 20-25° C. The zinc-fluoride composition may reduce bacteria at the tooth surface.

In some embodiment, the method for reducing bacteria on a tooth surface may further include any additional steps or features described above.

In some embodiments, the reduction of bacteria may be quantified or qualified by log reduction or zone inhibition, respectively. For example, the methods described herein may provide a log reduction of caries-causing bacteria about 1 to about 6. For example, the methods described herein may provide a zone inhibition of caries-causing bacteria of about 6 mm to about 20 mm.

In many embodiments, the methods described herein are especially suited for reducing bacteria on an interproximal tooth surface, i.e., between adjacent teeth.

### Remineralization

In one embodiment, a method for remineralizing a tooth surface is described. The method may include providing at least one applicator and a zinc-fluoride composition, contacting an effective amount of the zinc-fluoride composition to the tooth surface with the at least one applicator, and allowing the zinc-fluoride composition to contact the tooth surface for a period. The zinc-fluoride composition may include a zinc carboxylate, an amine-containing ligand, a source of fluoride anion effective to provide fluoride in an amount of at least 4 wt % with respect to the weight of the zinc-fluoride composition, and water. The zinc-fluoride composition may have a pH of at least 8 and be a homogenous solution at a temperature of about 20-25° C. The zinc-fluoride composition may remineralize the tooth surface.

In some embodiment, the method for remineralizing a tooth surface may further include any additional steps or features described above.

In some embodiments, the remineralization of the tooth surface may be quantified by an increase in hardness as measured by indentation hardness measurements with an indentor, such as Vickers (VHN) or Knoop (KHN). For example, the methods described herein may provide an increase in hardness of about VHN10 to about VHN350.

In many embodiments, the methods described herein are especially suited for remineralizing an interproximal tooth surface, i.e., between adjacent teeth.

### Kits

In one embodiment, a kit is provided. The kit may include a dental article including a at least one applicator and a zinc-fluoride composition. Alternatively, the kit may include at least one applicator and a zinc-fluoride composition. The zinc-fluoride composition may include a zinc carboxylate, an amine-containing ligand, a source of fluoride anion effective to provide fluoride in an amount of at least 4 wt % with respect to the weight of the zinc-fluoride composition, and water. The zinc-fluoride composition may have a pH of at least 8 and be a homogenous solution at a temperature of about 20-25° C. The kit may further include a set of instructions directing a user to perform method steps for reducing bacteria at a tooth surface described herein or methods steps for remineralizing a tooth surface described herein.

### EXAMPLES

Objects and advantages of this disclosure are further illustrated by the following examples, but the particular materials and amounts thereof recited in these examples, as well as other conditions and details, should not be construed to unduly limit this disclosure. These examples are merely for illustrative purposes only and are not meant to be limiting on the scope of the appended claims.

TABLE 1
Materials

| Description | Source | Location |
|---|---|---|
| Ammonium fluoride | Honeywell | Germany |
| Ammonia water solution 30% | J. T. Baker | Central valley, PA |
| BisGMA (2,2-bis[4-(2-hydroxy-3- | CAS Reg. No. 1565-94-2 | |
| methacryloyloxypropoxy)phenyl]propane) | | |
| HEMA (2-hydroxyethyl methacrylate) | ESSTECH, Inc. | Essington, PA, USA |
| IRGACURE 819 (CAS 162881-26-7) | Sigma Aldrich | St Louis, MO, USA |
| L-arginine, free base | MP biomedicals LLC | Solon, OH, USA |
| PERIDEX Chlorhexidine Gluconate | 3M ESPE | St Paul, MN, USA |
| 0.12% Oral Rinse | | |
| SDF, commercially available as | Elevate Oral Care | West Palm Beach, FL, USA |
| ADVANTAGE ARREST silver diamine | | |
| fluoride solution, 38% | | |
| Zinc citrate trihydrate | Jungbunzlauor | Basel, Switzerland |
| Zinc potassium fluoride | City Chemical LLC | West Haven, CT, USA |

### Examples EX1 to EX10 and Comparative Examples CE1 To CE4

All example components were added into a plastic centrifuge tube. Compositions were mixed well by a vortex mixer to form a suspension. Mixtures were then heated to 60° C. and continued to be mixed several times. Mixtures were kept in a 60° C. oven for 24 hours. Example mixtures were vortex mixed several times during warming in 60° C. oven to form solutions. Comparative examples (CE) did not form solution; the zinc citrate and zinc potassium fluoride could not dissolve in the solution and these comparative examples exhibited low water solubility compared with examples.

TABLE 2
EXAMPLES EX1 to EX5 and COMPARATIVE
EXAMPLES CE1 and CE2

| Components | EX1 | EX2 | EX3 | EX4 | EX5 | CE1 | CE2 |
|---|---|---|---|---|---|---|---|
| zinc citrate trihydrate | 41.7 | 43.5 | 40.8 | 40.0 | 29.4 | 46.5 | 0 |
| NH₃ water solution | 41.7 | 43.5 | 40.8 | 44.0 | 0 | 0 | 0 |
| 30% | | | | | | | |
| NH₄F | 16.7 | 13.0 | 18.4 | 16.0 | 11.8 | 0 | 0 |
| water | 0 | 0 | 0 | 0 | 29.4 | 53.5 | 48.3 |
| L-arginine | 0 | 0 | 0 | 0 | 29.4 | 0 | 51.7 |
| Zinc potassium | 0 | 0 | 0 | 0 | 0 | 0 | 0 |
| fluoride | | | | | | | |
| Total | 100 | 100 | 100 | 100 | 100 | 100 | 100 |
| pH (measured with | 10 | 10 | 10 | 10 | 9 | NA | NA |
| 1-14 pH paper) | | | | | | | |
| Zinc % | 12.9 | 13.4 | 12.7 | 12.4 | 9.1 | NA | NA |
| Fluoride % | 8.6 | 6.7 | 9.4 | 8.2 | 6.0 | NA | NA |
| Total water % | 29.1 | 30.4 | 28.6 | 30.8 | 29.4 | 53.5 | 51.7 |
| formed solution | Yes | Yes | Yes | Yes | Yes | No | No |
| after reaction at | | | | | | | |
| 60° C. | | | | | | | |

TABLE 3
EXAMPLES EX6 to EX10 and COMPARATIVE
EXAMPLES CE3 and CE4

| Components | EX6 | EX7 | EX8 | EX9 | EX10 | CE3 | CE4 |
|---|---|---|---|---|---|---|---|
| zinc citrate trihydrate | 25.8 | 26.5 | 27.6 | 24.2 | 21.6 | 34.8 | 0 |
| NH₃ water solution | 0 | 0 | 0 | 0 | 0 | 0 | 60.0 |
| 30% | | | | | | | |
| NH₄F | 9.7 | 9.9 | 10.3 | 9.1 | 8.1 | 13.0 | 0 |
| water | 38.7 | 39.7 | 34.5 | 42.4 | 48.7 | 52.2 | 0 |
| L-arginine | 25.8 | 23.8 | 27.6 | 24.2 | 21.6 | 0 | 0 |
| Zinc potassium | 0 | 0 | 0 | 0 | 0 | 0 | 40.0 |
| fluoride | | | | | | | |
| Total | 100 | 100 | 100 | 100 | 100 | 100 | 100 |
| pH (measured with | 9 | 9 | 9 | 9 | 9 | NA | NA |
| 1-14 pH paper) | | | | | | | |
| Zinc % | 8.0 | 8.2 | 8.6 | 7.5 | 6.7 | NA | NA |
| Fluoride % | 5.0 | 5.1 | 5.3 | 4.7 | 4.2 | NA | NA |
| Total water % | 38.7 | 39.7 | 34.5 | 42.4 | 48.7 | 52.2 | 42.0 |
| Formed solution | Yes | Yes | Yes | Yes | Yes | No | No |
| after reaction at | | | | | | | |
| 60° C. | | | | | | | |

### Remineralization and Acid Resistance Testing

Specimens of enamel were prepared from bovine teeth. Caries lesions were created in the enamel by immersing each specimen in a solution of 0.1M lactic acid and 0.2% CARBOPOL (pH=5) at 37° C. for 24 hours. The specimens were randomized to one of the following treatment liquids: artificial saliva (used as a control), silver diamine fluoride (SDF) as a comparative solution, EX1, EX2, and EX3. A total of 10 specimens per treatment solution were prepared.

The specimens were treated by applying the different test treatment liquids on the enamel lesion with a mini dental bush, rubbing the lesion with the mini dental brush for 10 seconds, waiting for one minute, rinsing with artificial saliva, and storing in artificial saliva for 30 minutes at 37° C. Specimens were then rinsed with artificial saliva again and placed in fresh artificial saliva for 24 hours at 37° C.

The surface hardness (Vickers Hardness Number (VHN)) of treated specimens was measured by a Vickers hardness tool. The average VHN and standard deviation of the 10 specimens per treatment were calculated.

To measure the retention of the surface micro-hardness, the specimens were next placed in a solution of 0.1M lactic acid and 0.2% CARBOPOL (pH=5) for 24 hours to create a demineralization challenge. The retention of any increased hardness was evaluated via acid challenge. Samples were subjected to demineralization solution for 24 hours. Retention of micro-hardness following the acid challenge indicates an ability of the treatment to resist demineralization. Surface micro-hardness was measured following the acid challenge. Micro-hardness values that were higher than baseline measurements indicated remineralization of tooth enamel by the treatment. The specimens were stored in artificial saliva for 24 hours at 37° C., and then hardness was measured. The specimens were acid challenged by demineralization solution and the hardness were measured again. The more resistant the enamel to the challenge, the higher the retention of the surface micro-hardness value. Surface micro-hardness was measured using the same procedure as with the baseline measurement. Any increase in micro-hardness after treatment indicates an ability of the treatment solution to remineralize tooth enamel.

TABLE 4
Enamel Micro-hardness (VHN) and
Remineralization Testing Results

| | Saliva | | | | |
|---|---|---|---|---|---|
| Treatment | Control | SDF | EX1 | EX2 | EX3 |
| Baseline Average | 45.6 | 45.9 | 46.8 | 47.3 | 47.7 |
| Baseline Std. Dev. | 7.1 | 7.3 | 7.3 | 7.2 | 7.2 |
| Treatment Average | 61.5 | 75.8 | 81 | 97.5 | 102.4 |
| Treatment Std. Dev. | 12.5 | 16.4 | 11.8 | 19.1 | 19.5 |
| Acid Challenge Average | 46.1 | 68.2 | 55.1 | 72.8 | 77.1 |
| Acid Challenge Std. Dev. | 9.9 | 16.3 | 9.5 | 16.1 | 22.5 |
| Saliva Remin. Average | 57 | 88.0 | 84.2 | 92.9 | 115.6 |
| Saliva Remin. Std. Dev. | 12.7 | 22.3 | 10.1 | 22.3 | 23.9 |
| Second Acid Challenge Average | 42.2 | 68.4 | 54.9 | 73.9 | 80.8 |
| Second Acid Challenge Std. Dev. | 8.5 | 15.7 | 10.3 | 15.8 | 21.7 |

### Fluoride Uptake Testing

Specimens of enamel were prepared from bovine teeth. Caries lesions were created in the enamel by immersing each specimen in a solution of 0.1M lactic acid and 0.2% CARBOPOL (pH=5) at 37° C. for 24 hours. The specimens were randomized to one of the following treatment liquids: artificial saliva (used as a control), silver diamine fluoride (SDF) used as a comparison, EX1, EX2, and EX3. A total of 10 specimens per treatment group were prepared.

The specimens were treated by applying the different test treatment liquids on the enamel lesion with a mini dental bush, rubbing the lesion with the mini dental brush for 10 seconds, waiting for one minute, rinsing with artificial saliva, and storing in artificial saliva for 30 minutes at 37° C. Specimens were then rinsed with artificial saliva again and placed in fresh artificial saliva for 24 hours at 37° C.

Microdrill biopsies were taken of each specimen to measure the amount of fluoride transferred to the enamel. To test the fluoride incorporation into the tooth, specimens were further subjected to an acid challenge. Specimens were placed into a solution of 0.1M lactic acid and 0.2% CARBOPOL (pH=5) at 37° C. for 24 hours to simulate an acid challenge. Microdrill biopsies were taken from each specimen to measure the amount of fluoride in the enamel following acid challenge. Results in units of μg F/cm² are reported in Table 5.

TABLE 5
Fluoride Uptake (μg F/cm²) After Example Solution Treatments

| | | | Acid | |
|---|---|---|---|---|
| | Treatment | | Challenge | Acid |
| | Average | Treatment | Average | Challenge |
| Treatment | (μg F/cm²) | Std. Dev. | (μg F/cm²) | Std. Dev. |
| Saliva control | 2.29 | 1.01 | 3.38 | 1.50 |
| SDF | 16.44 | 3.21 | 14.07 | 1.68 |
| EX1 | 15.46 | 3.20 | 8.72 | 3.03 |
| EX2 | 10.34 | 4.41 | 7.93 | 2.21 |
| EX3 | 14.22 | 2.93 | 12.26 | 3.56 |

### Examples EX11 to EX13 and Comparative Example CE5

Additional examples were prepared using commercially available SDF in combination with the zinc complex solution of example EX3. The following examples illustrate the use of example compositions on tooth surfaces. Bovine teeth samples were prepared in the following manner to serve as a test surface for example treatments. Bovine teeth were held in an acrylic mold and polished with 120 grit sand paper to expose the dentin, then polished with 320 grit sandpaper to smooth the dentin surface. One drop of each example composition was placed on a prepared bovine dentin surface and then exposed to a blue LED light using 3M ELIPAR DEEPCURE-S LED curing light, (available from 3M Company of St. Paul, Minn., USA) with maximum wavelength 450 nm and output approximately 1500 mW/cm² for 20 seconds. Examples EX11-EX13 demonstrates that while silver diamine fluoride can be used as an additional source of fluoride and will have the antimicrobial benefit of zinc in the composition, the final condition is less aesthetically pleasing than examples that do not turn black when exposed to light, such as EX3.

TABLE 6
EXAMPLES EX11 to EX13 and
COMPARATIVE EXAMPLE CE5

| Components | EX11 | EX12 | EX13 | EX3 | CE5 |
|---|---|---|---|---|---|
| EX3 | 76.9 | 62.5 | 50 | 100 | 0 |
| SDF | 23.1 | 37.5 | 50 | 0 | 100 |
| Total | 100 | 100 | 100 | 100 | 100 |
| Formed solution | Yes | Yes | Yes | Yes | Yes |
| Turned black on tooth | Yes | Yes | Yes | No | Yes |
| after blue light exposure | | | | | |

### Examples EX14 and Comparative Example CE6

Additional examples were prepared by combining the zinc fluoride complex of EX3 or SDF with a photo-curable methacrylate mixture.

TABLE 7
EXAMPLE EX14 and COMPARATIVE EXAMPLE CE6

| Components | EX14 | CE6 |
|---|---|---|
| EX3 | 28.5 | 0 |
| SDF | 0 | 9.1 |
| HEMA | 35.4 | 45 |
| BisGMA | 35.4 | 45 |
| IRGACURE 819 | 0.7 | 0.9 |
| Total | 100 | 100 |
| Cured into hard material | Yes | Yes |
| after light exposure | | |
| Turned black on tooth | No | Yes |
| after light exposure | | |

### Zone of Inhibition Antimicrobial Test

A multispecies zone of inhibition test was performed according to the following steps in order to assess antimicrobial efficacy of examples. Human saliva was used as the source of bacteria.
1. Sterilized 6 mm diameter round filter paper disks were used.
2. Experimental and control solutions were dispensed into standard 96 well plates in preparation to soak the filter paper disks.
3. Each sterilized paper disk was soaked by applying 10 μL of corresponding solution to the disks. Each sample was done in triplicate and final results were averaged.
4. Human saliva and mucin containing medium mixture was spread on the agar plate. Agar plates were divided into equal sections according to the number of solutions to be tested. Sections were marked with the names of the test solutions.
5. The agar plate lid was lifted off and the soaked filter paper samples were place onto the agar. Each sample filter paper disk was then gently pushed down to ensure complete contact of the filter paper disk with the agar surface. Caution was taken to not move a disk once it contacted the agar surface.
6. The analyst continued to place one sample disk at a time onto the agar surface until all disks were placed into their respective sections of each agar plate, as described above.
7. Once all disks were in place, the lid was replaced, the agar plates were inverted, and placed in a 37° C. air incubator for 24 hours.
8. After 24 hours incubation at 37° C., the diameter of each zone of inhibition was measured to the nearest millimeter using a ruler or calipers. The zones of inhibition were measured as the diameter from the edges of the last visible colony, according to the unaided eye.
9. During measurements, care was taken to not touch the disk or surface of the agar. The ruler was decontaminated between each measurement; measurements were performed in a biosafety cabinet.

TABLE 8
Zone of Inhibition Antimicrobial Testing Results

| | Zone of Inhibition | Std. Dev. |
|---|---|---|
| Sample | Diameter Ave. (mm) | (n = 3) |
| 0.9% Saline Solution | 6 | 0 |
| CHG Rinse | 11 | 1 |
| SDF | 13 | 0 |
| EX1 | 17 | 2 |
| EX2 | 18 | 0 |
| EX3 | 17 | 4 |
| EX4 | 17 | 1 |
| EX11 | 14 | 1 |
| EX12 | 14 | 2 |
| EX13 | 14 | 1 |

### Accelerated Weight Loss Testing in Foil Pouch

### Example EX15

Additional examples may be prepared in the following manner. ORAL-B SUPER FLOSS may be selected as the example applicator. The ORAL-B SUPER FLOSS (commercially available from Proctor & Gamble Company of Cincinnati Ohio, USA) has a stiffened-end dental floss threader portion, a spongy floss portion, and a regular floss portion. The spongy floss portion of the ORAL-B SUPER FLOSS is an absorbent portion of the applicator. The spongy floss portion of the ORAL-B SUPER FLOSS may be dipped into a solution of any representative example of present disclosure. The entire ORAL-B SUPER FLOSS, soaked with the representative example, may be placed in an aluminum foil pouch (Oliver Pouch, part #87891, 10 cm 20 cm; commercially available from Oliver Healthcare Packaging, Inc.). The foil pouch may then be sealed with, for example, a hand-pressed Impulse sealer, such as model type A1E-300, with 300 watts AC120V 60 Hz, available from American International Electric Sealer Supply. Representative examples loaded onto an applicator as described above, and sealed in an aluminum pouch, represent single use packages of one embodiment of the present disclosure. Such packaged examples may then be stored for extended periods of time in temperature-controlled units (ovens); for example for one or two months at 60° C. Properly sealed units would exhibit minimal or no weight loss over those intervals and be considered acceptable. Storage at 60° C. for 2 months is generally considered to be equivalent to 2 years storage at room temperature.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A dental article configured to access an interproximal gap, the article comprising:
at least one applicator;
zinc-fluoride composition comprising:
a zinc carboxylate;
an amine-containing ligand;
a source of fluoride anion effective to provide fluoride in an amount of at least 4 wt % with respect to the weight of the zinc-fluoride composition; and
water,
wherein:
the zinc-fluoride composition has a pH of at least 8, and
the zinc-fluoride composition is a homogenous solution at a temperature of about 20-25 ° C.; and
a package for containing one or more of the at least one applicator and the zinc-fluoride composition, wherein the package is impermeable to water vapor.

2. (canceled)

3. The dental article of claim 1, wherein the applicator is selected from a floss, a floss pick, a pick, a microbrush, a swab, or a combination thereof.

4. The dental article of claim 1, wherein the applicator is pre-saturated with the zinc-fluoride composition.

5. The dental article of claim 1, wherein the zinc-fluoride composition consists essentially of the zinc carboxylate, the amine-containing ligand, the source of fluoride, and water.

6. The dental article of claim 1, wherein the zinc carboxylate is selected from zinc citrate, zinc gluconate, zinc maleate, zinc acetate, zinc fumarate, zinc adipate, zinc propionate, hydrates thereof, and combinations thereof.

7. The dental article of claim 1, wherein the zinc carboxylate is zinc citrate.

8. The dental article of claim 1, wherein the amine-containing ligand is selected from one or more of an amino acid and ammonia.

9. The dental article of claim 1, wherein the amine-containing ligand is an amino acid selected from histidine, isoleucine, leucine, lysine, tyrosine, tryptophan, methionine, phenylalanine, aspartic acid, glycine, arginine, glutamic acid, valine, alanine, threonine, cysteine, proline, asparagine, and combinations thereof.

10. (canceled)

11. The dental article of claim 1, wherein the amine-containing ligand is ammonia.

12. The dental article of claim 1, wherein the source of fluoride anion is selected from sodium fluoride, ammonium fluoride, silver fluoride, stannous fluoride, potassium fluoride, and combinations thereof.

13. (canceled)

14. The dental article of claim 1, wherein the amine-containing ligand is present in amount between about 5 wt % and about 30 wt % with respect to the weight of the zinc-fluoride composition.

15. The dental article of claim 1, wherein the zinc carboxylate is present in an amount effective to provide zinc in about 5 wt % to about 15 wt % with respect to the weight of the zinc-fluoride composition.

16. The dental article of claim 1, wherein the water is present in an amount of about 20 wt % to about 50 wt % with respect to the weight of the zinc-fluoride composition.

17. The dental article of claim 1, wherein the amine-containing ligand is present in amount of about 10 wt % to about 15 wt % with respect to the zinc-fluoride composition, and provides zinc:amine-containing ligand mol ratio of about 1:2 to about 1:3.

18. A method for one or more of reducing bacteria on a tooth surface and remineralizing a tooth surface, the method comprising:
providing a dental article of claim 1;
contacting an effective amount of the zinc-fluoride composition to the tooth surface with the at least one applicator; and
allowing the zinc-fluoride composition to contact the tooth surface for a period,
wherein the zinc-fluoride composition one or more of reduces the bacteria at the tooth surface and remineralizes the tooth surface.

19. The method of claim 18, further comprising contacting the at least one applicator to the tooth surface and loading the applicator with the zinc-fluoride composition.

20. The method of claim 18, wherein the at least one applicator is pre-loaded with the zinc-fluoride composition.

21. (canceled)

22. The method of claim 18, wherein the effective amount is at least about 30 mg of zinc-fluoride composition.

23. A method of claim 18, wherein the tooth surface is an interproximal tooth surface.

24. A kit comprising:
a dental article of claim 1, or
at least one applicator, and
zinc-fluoride composition comprising:
a zinc carboxylate;
an amine-containing ligand;
a source of fluoride anion effective to provide fluoride in an amount of at least 4 wt % with respect to the weight of the zinc-fluoride composition; and
water,
wherein:
the zinc-fluoride composition has a pH of at least 8, and
the zinc-fluoride composition is a homogenous solution at a temperature of about 20-25° C.; and
a set of instructions directing a user to contact an effective amount of the zinc-fluoride composition to the tooth surface with the at least one applicator.
